(19) United States
(12) Patent Application Publication
(10) Pub. No.: US 2022/0087964 A1
(43) Pub. Date: Mar. 24, 2022

(54) MATERNAL DPA FOR BENEFIT OF MOTHER AND/OR CHILD'S MENTAL HEALTH

(71) Applicants: AGENCY FOR SCIENCE, TECHNOLOGY AND RESEARCH (A*STAR), Singapore (SG); NATIONAL UNIVERSITY OF SINGAPORE, Singapore (SG)

(72) Inventors: Neerja KARNANI, Singapore (SG); Gerard Kum Peng WONG, Singapore (SG); Peter David GLUCKMAN, Auckland (NZ); Michael Joseph MEANEY, Beaconsfield, Quebec (CA)

(21) Appl. No.: 17/421,540

(22) PCT Filed: Jan. 23, 2020
(86) PCT No.: PCT/SG2020/050035

§ 371 Date: Jul. 8, 2021

(30) Foreign Application Priority Data
Jan. 23, 2019 (SG) 10201900614P

(51) Int. Cl. A61K 31/202; A61K 9/20; A61P 25/24

(52) U.S. Cl. CPC A61K 31/202; A61P 25/24; A61K 9/20

(57) ABSTRACT

Interpretation: This is the first ever report to uncover a tangible lipidomic basis by which maternal emotional well-being may influence fetal neurodevelopment and the later risk for psychopathology. This novel insight points to the potential utility of nutritional approaches among pregnant women with high levels of depressive symptoms in the prevention of offspring risk for later socio-emotional behavioral problems predictive of future psychopathology. Such a novel approach is particularly pertinent in light of the preference to avoid pharmacological interventions such as anti-depressant medications during pregnancy.

### BRIEF DESCRIPTION OF THE INVENTION

This invention relates to the prenatal administration of polyunsaturated fatty acids (PUFAs), preferably including docosapentaeonic acid (DPA) to protect a child against adverse mental wellness, and to protect the mother against depression, including post-partum depression. Also this invention pertains to formulations comprising DPA as an active ingredient; and compositions which comprise DPA and another active ingredient(s) of benefit to a pregnant woman.

### BACKGROUND OF THE INVENTION

The various polyunsaturated fatty acids (PUFAs) play nutritional roles throughout the lifetime.

While the roles of DHA and EPA have largely been investigated, much less is known about DPA. Li et al 2016 Eur J Lipid Sci Tech. 118:1692-1701 reviews many aspects, including the observation that lower blood levels of DPA are associated with an increased incidence of mental health disorders, including depression. DPA is also known to have an anti-inflammatory effect on brain tissue. It is suggested that DPA supplementation may improve the mental health of a mother, and ease post-natal depression.

Miller et al 2013 Eur J Nutr 52:895-904 investigated the role of docosapentaenoic acid (DPA) in women. They found that DPA could metabolize to both EPA and DHA.

It would be desirable to supplement the maternal diet so that the offspring benefit, particularly in the area of mental health.

### DETAILED DESCRIPTION OF THE INVENTION

We have found, according to our invention, clear evidence of the relationship between maternal emotional well-being, the placental lipidome and child socio-emotional development, suggesting that the mood-associated variation in nutrient status of the mother affects availability to the fetus. Our longitudinal analysis intricately identifies a subset of maternal mood-associated placental lipids that are also associated with socio-emotional outcomes in the offspring. Reduced levels of key placental lipids also corroborated with low DPA levels in maternal plasma at 26 weeks of gestation, a time point at which antenatal depression was assessed. These findings suggest that nutritional manipulation may be an effective intervention in women with antenatal depression in relation to the prevention of subsequent sub-optimal neurodevelopment in the offspring.

Thus one embodiment of this invention is a method of decreasing the risk, preventing, or amelioration the symptoms of depression in a woman during pregnancy comprising administering an effective amount of DPA during her pregnancy.

Another embodiment of our invention is a method of enhancing the socio-emotional outcome of a child by antenatally administering to the child's mother a composition comprising an effective amount of a DPA and optionally at least one other PUFA selected from the group consisting of EPA and DHA and an excipient. Preferably, the PUFA comprises an effective amount of DPA. In some embodiments, the child which has an enhanced socio-emotional outcome is at least 4 years old. In some embodiments, the socio-emotional outcome is seen in early childhood, or up to age 5.

Another embodiment is a method of modifying the placental lipid makeup comprising administering an effective amount of at least one PUFA comprising DPA to a pregnant woman during her pregnancy. This has the effect of modifying the lipid environment of the unborn child, and the effect is observed in that the child experiences an enhanced socio-emotional outcome.

In some embodiments the PUFAS, preferably comprising an effective amount of DPA are administered during at least the third trimester of pregnancy, more preferably during the second and third trimester, or during at least part of the first trimester and during the second and third trimesters. In some embodiments, the PUFAs are administered as soon as the woman is aware that she is pregnant or are prescribed by the doctor at the first antenatal visit. In some embodiments they are taken prior to the woman becoming pregnant, so that they are administered throughout the entire pregnancy.

In other embodiments, the mother of the child is not suffering from prenatal depression nor experiences symptoms or attributes associated with depression. In other embodiments, the mother of the child is at risk of suffering from pre- or post-natal depression (for example, this was experienced in prior pregnancies, or her general nutrition and PUFA levels are lower than desired, or she is at risk due to genetic factors (e.g. a family history of psychiatric disorders, or having experienced non-pregnancy-related depression) or her socio-economic status.

Another embodiment of our invention is a maternal nutritional supplement comprising DPA in a tablet or capsule form. DPA is not currently commercially available in a pure form. However, this invention also relates to the use of combinations of DPA and another PUFA, such as docosahexaenoic acid (DHA) and/or eicosapentaenoic acid (EPA). Commercially available forms of DPA (concentration of 20-60%) blended with either EPA and DHA, are sold by KDNutra, Pennsylvania, USA.

Another embodiment of this invention is a composition comprising DPA and DHA, where the weight ratio of DPA to DHA is from 40-60 to 60-40. Another embodiment is a composition comprising DPA and EPA, where the weight ratio of DPA to EPA is from 40-60 to 60-40. Another embodiment of this invention is a composition comprising 10% by weight of DPA.

Yet another embodiment of this invention is a tablet comprising DPA and at least one excipient selected from the group consisting of: sugars, modified starch, binders and other known processing aids.

In accordance with this invention, we identified an association between maternal depressive symptoms and multiple placental lipid species, particularly phospholipids containing LC-PUFAs. Analysis of maternal plasma and placental samples revealed a strong congruence in lipid levels between the maternal and feto-placental compartments, consistent with maternal circulating lipid levels being the major determinant of placental lipid uptake and subsequent transfer to the fetus. Finally, our prospective, longitudinal analysis confirmed the established association between antenatal maternal depressive symptoms and socio-emotional dysfunction in the offspring. We also showed that specific placental phospholipids significantly associated with maternal mood were also independently associated with socio-emotional function in the offspring. These findings provide novel evidence for the role of phospholipids in the mother-offspring shared placental environment, on antenatal maternal mental health and future socio-emotional development of the offspring.

### Definitions

As used throughout this text and claims, the following abbreviations will be used:
PUFA: polyunsaturated fatty acid
DHA: Docosahexaenoic acid, and includes its derivatives, such as ethers, and salts.
EPA: Eicosapentaenoic acid, and includes its derivatives, such as ethers, and salts
DPA: Docosapentaeonic Acid, and includes its derivatives, such as ethers and salts
AA: Arachidonic Acid, and includes its derivatives, such as ethers and salts
DSM: The Diagnostic and Statistical Manual of Mental Disorders, 5^{th} Edition, published by the American Psychiatric Association.
EPDS: The Edinburgh Postnatal Depression Scale—this is the most commonly used validated tool to screen for depression in pregnancy and post-partum periods.
Socio-emotional outcome: this is assessed and measured by the Child Behavior Checklist, a widely used, validated caregiver report which is used to identify problem behavior in children. It is a component of the Achenbach System of Empirically Based Assessment. It looks at eight syndromes: Aggressive Behavior, Anxious/Depressed, Attention Problems, Rule-Breaking Behavior, Somatic Complaints, Social Problems, Thought Problems, and Withdrawn/Depressed.
   Internalizing Problems according to Child Behavior Checklist (CBCL) includes anxious/depressed, withdrawn-depressed, and somatic complaints
   Externalizing problems according to CBCL includes items related to physical aggression, disobeying rules, and disruptive, hyperactive and aggressive behavior
   Total problems according to CBCL is the sum of the scores of all the problem items
   Syndrome Scales of the Child Behavior Checklist:
Emotionally reactive
Anxious-depressed
Withdrawn behavior
Attention problems
Aggressive behavior
Sleep problems
Somatic complaints
   DSM-Oriented Scales: These scales comprise CBCL items that experienced psychiatrists and psychologists from many cultures rated as being very consistent with DSM-5 diagnostic categories:
      Depressive Problems;
      Anxiety Problems;
      Autism Spectrum Problems;
      Attention Deficit/Hyperactivity Problems;
Oppositional Defiant Problems.
“Enhancing socio-emotional outcome” means that the component measured in a child having received the antenatal PUFA is higher than that measured in the population of children who have not received the antenatal PUFA.

### BRIEF DESCRIPTION OF THE FIGURES

FIG. 1 is a chart summarizing the correlations observed between maternal lipid levels and mental health aspects of the child at 48 months.

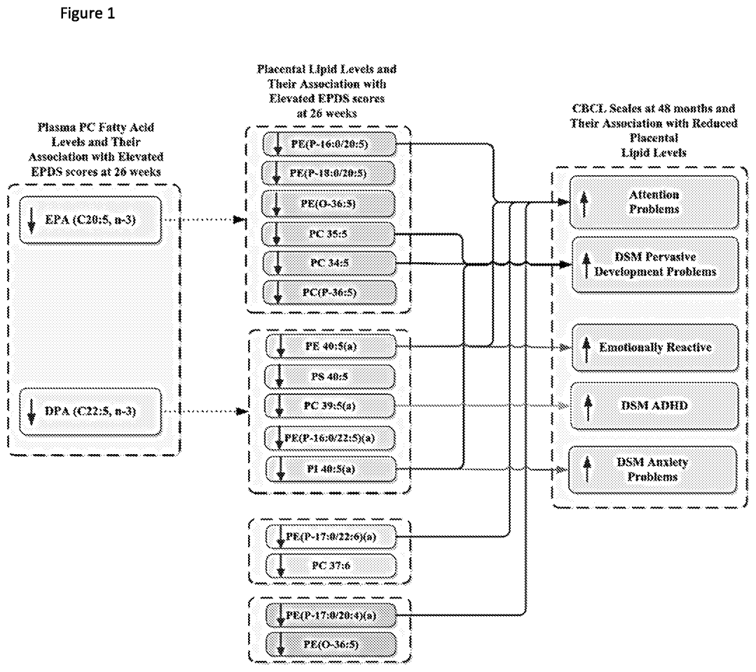

FIG. 2 is a scatterplot comparing beta-coefficients of 57 fetal-facing placenta and corresponding maternal-facing placenta samples in multiple adjusted linear regression analysis with EPDS at 26 weeks as the outcome. A significant linear correlation (rho=0.843, p=3e−129) is observed.

FIG. 3 is a table showing the association of placental lipid levels with CBCL scales at 48 months in the offspring as determined by multiple adjusted linear regression without EPDS as a covariate (first 8 CBCL scales shown).

FIG. 4 is a table showing the association of placental lipid levels with CBCL scales at 48 months in the offspring as determined by multiple adjusted linear regression without EPDS as a covariate (last 7 CBCL scales shown).

We found that there is an association between low DPA levels in the placenta and depression in the mother (both pre- and post natally). This was particularly observed in women expressing mild or sub-clinical depressive symptoms. Thus another embodiment of this invention is a method of enhancing a low mood of a peri-partum woman comprising administration of an effective amount of DPA to the woman. The enhancement of her mood can be measured by the administration of the EPDS test, so that the score achieved on the EPDS test registers in the upper two-thirds or higher of the scoring system, which is considered in the “normal” or “above normal” scoring category. Another embodiment of this invention is the non-therapeutic peri-partum use of DPA to enhance mood of a woman. Yet another embodiment of this invention is the use of DPA in the manufacture of a nutritional supplement or pharmaceutical to enhance the mood of a peri-partum woman.

We also found that there is a direct association between the lipid levels found in the placenta and the socio-emotional outcome of the child measured at 48 months, as detailed below. Lower placenta levels of DPA, EPA, AA and DHA were significantly associated with the Attention Problems scale. Thus, one embodiment of this invention is a method of enhancing a child's socio-emotional outcome as related to attention problems comprising antenatally administering to the child's mother an effective amount of at least one PUFA selected from the group consisting of DPA, EPA, AA and DHA. Another embodiment of this invention is the antenatal non-therapeutic use of at least one PUFA selected from the group consisting of DPA, EPA, AA and DHA to improve a child's socio-emotional outcome as related to attention problems. Yet another embodiment of this invention is the use of a PUFA selected from the group consisting of DPA, EPA, AA and DHA in the manufacture of a nutritional supplement or pharmaceutical to improve the socio-emotional outcome of a child as related to attention problems.

It was also found in accordance with our invention that low placental EPA and DPA levels were each significantly associated with the socio-emotional outcome of DSM Pervasive Developmental Problems scale. Pervasive developmental problems characterized by delays in the development of socialization and communication skills. Symptoms may include problems with using and understanding language, difficulty relating to people, objects and events; unusual play with toys and other objects; difficulty with changes in routine or familiar surroundings, and repetitive body movements or behavior patterns. Autism is the most characteristic and best studied pervasive developmental disorder. Thus, one embodiment of this invention is a method of enhancing a child's socio-emotional outcome as related to a Pervasive Developmental problem comprising antenatally administering to the child's mother an effective amount of at least one PUFA selected from the group consisting of DPA and EPA. Another embodiment of this invention is the non-therapeutic antenatal use of at least one PUFA selected from the group consisting of DPA, and EPA, AA to improve a child's socio-emotional outcome as related to Pervasive Developmental problem. Yet another embodiment of this invention is the use of a PUFA selected from the group consisting of DPA, and EPA, in the manufacture of a nutritional supplement or pharmaceutical to improve the socio-emotional outcome of a child as related to a Pervasive Developmental Problem.

We have also found that a lower placental DPA was significantly associated with the Emotionally Reactive scale. Emotional reactivity refers to the tendency to experience frequent and intense emotional arousal. Thus another embodiment of this invention is a method of enhancing a child's socio-emotional outcome as related to emotionally reactivity problems comprising antenatally administering to the child's mother an effective amount of DPA. Another embodiment of this invention is the non-therapeutic antenatal use of DPA to improve a child's socio-emotional outcome as related to emotional reactivity problems. Yet another embodiment of this invention is the use of DPA in the manufacture of a nutritional supplement or pharmaceutical to improve the socio-emotional outcome of a child as related to emotional reactivity problems.

We have also found that low placental DPA was significantly associated with the DSM Anxiety Problems scale. Symptoms include the presence of excessive anxiety and worry about a variety of topics, events or activities, even if there is no specific threat. The worrying is accompanied by at least one of: edginess or restlessness, tiring easily; more fatigued than usual, impaired concentration or feeling as though the mind goes blank, irritability (which may or may not be observable to others), increased muscle aches or soreness, difficulty sleeping (due to trouble falling asleep or staying asleep, restlessness at night, or unsatisfying sleep). Thus another embodiment of this invention is a method of enhancing a child's socio-emotional outcome as related to anxiety comprising antenatally administering to the child's mother an effective amount of DPA. Another embodiment of this invention is the non-therapeutic antenatal use of DPA to improve a child's socio-emotional outcome as related to anxiety. Yet another embodiment of this invention is the use of DPA in the manufacture of a nutritional supplement or pharmaceutical to improve the socio-emotional outcome of a child as related to anxiety.

We have also found that low placental DPA was significantly associated with the DSM ADHD scale. ADHD is considered to have three component features: inattention, hyperactivity, and impulsivity, although an individual's manifestation of each of these components may vary and change across the individual's lifespan. Thus, another embodiment of this invention is a method of enhancing a child's socio-emotional outcome as related to at least one component selected from the group consisting of: inattention, hyperactivity, and impulsivity comprising antenatally administering to the child's mother an effective amount of DPA. Another embodiment of this invention is the non-therapeutic antenatal use of DPA to improve a child's socio-emotional outcome as related to at least one component selected from the group consisting of: inattention, hyperactivity, and impulsivity. Yet another embodiment of this invention is the use of DPA in the manufacture of a nutritional supplement or pharmaceutical to improve the socio-emotional outcome of a child as related to at least one component selected from the group consisting of: inattention, hyperactivity, and impulsivity.

Formulations

The term “nutraceutical” as used herein denotes usefulness in both nutritional and pharmaceutical fields of application. Thus, nutraceutical compositions comprising EPA, DPA, AA, DHA and combinations thereof can be used as supplements to food and beverages or in various oral formulations, and as pharmaceutical formulations for enteral or parenteral application which may be solid formulations, such as capsules or tablets, or liquid formulations, such as solutions or suspensions.

The nutraceutical compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, gelling agents, gel-forming agents, antioxidants and antimicrobials.

The nutraceutical compositions according to the present invention may be in any galenic oral form containing a conventional carrier material that is suitable for administering to the body, e.g. in solid forms such as (additives/supplements for) food, food premix, fortified food, tablets, pills, granules, dragées, capsules and effervescent formulations, such as powders and tablets, or in liquid forms, such as solutions, emulsions or suspensions as e.g. beverages, pastes and oily suspensions. The pastes may be incorporated in hard- or soft-shell capsules, whereby the capsules feature e.g. a matrix of animal-derived gelatin, plant proteins or ligninsulfonate.

Examples of food are dairy products including, for example, margarines, spreads, butter, cheese, yoghurts or milk-drinks. Examples of fortified food include bread, cereal bars, bakery items, such as cakes and cookies, and potato chips or crisps. Beverages encompass non-alcoholic and alcoholic drinks as well as liquid preparations to be added to drinking water and liquid food. Non-alcoholic drinks are e.g., soft drinks, sports drinks, fruit juices, lemonades, teas and milk-based drinks. Liquid foods are e.g., soups and dairy products. The nutraceutical compositions containing EPA, DPA, DHA and combinations thereof may be added to a soft drink, an energy bar, or a candy.

If the nutraceutical composition is a pharmaceutical formulation the composition further contains pharmaceutically acceptable excipients, diluents or adjuvants. Standard techniques may be used for their formulation, as e.g. disclosed in Remington's Pharmaceutical Sciences, 20th edition Williams & Wilkins, PA, USA. For oral administration, tablets and capsules are preferably used which contain a suitable binding agent, e.g. gelatine or polyvinyl pyrrolidone, a suitable filler, e.g. lactose or starch, a suitable lubricant, e.g. magnesium stearate, and optionally further additives.

Dosages

The amount of DPA which should be administered per day to the average 70 kg person should range from about 1 to 50 mg, preferably from about 2 to 25 mg, and more preferably from 5 to 10 mg. In some embodiments it is 5 mg, and in some embodiments it is 10 mg per day. The dosages may be divided into partial doses and administered throughout the day, such as twice or three times per day if convenient.

The daily amount of DHA and/or EPA (by weight percent) is less critical, and is may be higher than the DPA. In some embodiments it is present in an amount of from 0.40 to 75 mg per day, more preferably from 0.8 mg to 37.5 mg per day, and more preferably from 2 mg to 60 mg per day. Again, the dosage can be in a single form, or divided up for multiple administrations during a day.

The non-limiting examples are presented to further illustrate the invention.

### Example 1

### The Clinical Trial

Methods

Study Design and Participants

Growing Up in Singapore Towards healthy Outcomes (GUSTO) 9 is a prospective birth cohort study involving deep phenotyping of pregnant women and their offspring. Written informed consent was obtained from all study participants. Briefly, the GUSTO study was designed to investigate the developmental origins of individual differences in multiple health outcomes. Pregnant women in their first trimester and of 218 years of age were recruited from the two major public hospitals in Singapore [KK Women's and Children's Hospital (KKH) and the National University Hospital (NUH)]. This study was ethically approved by the Domain-Specific Review Board of NUH and the Centralized Institutional Review Board of KKH. Eligible participants had to hold Singapore citizenship or permanent residency, or intent to reside in Singapore for the next five years, were of Asian ethnicity, and willing to contribute biosamples.

Of the 1247 recruited pregnant women in GUSTO, 1048 had placenta collected at delivery. All available placenta samples underwent targeted lipidomic analysis from which we selected subjects (N=70) of Chinese and Malay ethnicity with low levels of depressive symptoms (EPDS scores of less than 2) and subjects (N=116) with high levels of depressive symptoms (EPDS scores in the subclinical/clinical range of depression of more than 10) at 26-28 weeks gestation. Maternal age, ethnic composition, parity, pre-conception BMI were not statistically significant between the groups of subjects. Pregnancies conceived by in vitro fertilization and multiple pregnancies were excluded from this study.

Psychological Assessments

The Edinburgh Postnatal Depression Scale (EPDS) questionnaire was administered to mothers at 26 weeks of pregnancy to quantify levels of maternal depressive symptomatology. The EPDS is a widely used 10-item self-report scale designed as a screening instrument for postnatal depression validated for use in prenatal and postnatal depression.” Each item of the EPDS was scored on a four-point scale (0-3). The reliability of the EPDS score was 0.82 assessed using Cronbach's analysis for our cohort. Higher EPDS scores indicate a greater number of depressive symptoms.

When children were 48 months of age, parents completed the Child Behavior Checklist (CBCL/1.5-5), a parents' questionnaire that contains 99 problem items rating various scales reflecting dimensions of child socio-emotional function spanning categories of emotionally reactive, anxious/depressed, somatic complaints, withdrawn, sleep problems, attention problems, aggressive behavior, internalizing problems, externalizing problems and five DSM-oriented scales namely DSM anxiety problems, DSM affective problems, DSM pervasive developmental problems, DSM oppositional defiant problems and DSM attention-deficit/hyperactivity disorder (ADHD). The CBCL was validated and showed high reliability in Singaporean children. Previous studies show that antenatal maternal depressive symptoms associates with internalising scores in children in community samples.

Lipidomics of Placental Samples

Maternal and fetal-facing sides of placenta were collected, stored and homogenized. Briefly, lipids were extracted from 20 μL (approximately 100 μm of protein) of placental homogenate using chloroform/methanol (2:1, 20 volumes). Lipid analysis was performed by liquid chromatography, electrospray ionisation-tandem mass spectrometry using an Agilent 1290 liquid chromatography system with a 50 mm Zorbax Eclipse Plus I.8 μm C18 column, combined with an Agilent 6490 triple quadrupole mass spectrometer. The relative concentration of each lipid species was calculated from the area of the resultant chromatograms for the lipid species and the corresponding internal standards. A pilot study consisting of 57 maternal-facing placenta samples and 57 fetal-facing placenta samples were run initially to assess potential biases in lipidomic profiles arising from different sampling positions. 1048 maternal-facing placenta samples were subsequently analysed across 7 batches in the main experimental run.

Analysis of Fatty Acids in Plasma Phosphatidylcholine

Maternal fasting blood samples collected at 26-28 weeks' gestation were processed within 4 hours and stored at −80° C. Plasma lipids were extracted using chloroform-methanol (2:1, v/v), and phosphotidylcholine (PC) was isolated by solid phase extraction. Fatty acid methyl esters (FAME) were generated from PC after reaction with methanol containing 2% (v/v) sulphuric acid, extracted into hexane and separated by gas chromatography (GC). FAME were identified by comparison with retention times of previous standard runs and quantified using ChemStation software (Agilent Technologies). The absolute fatty acid concentration measurements were expressed as μg/mL of plasma.

Statistical Analysis

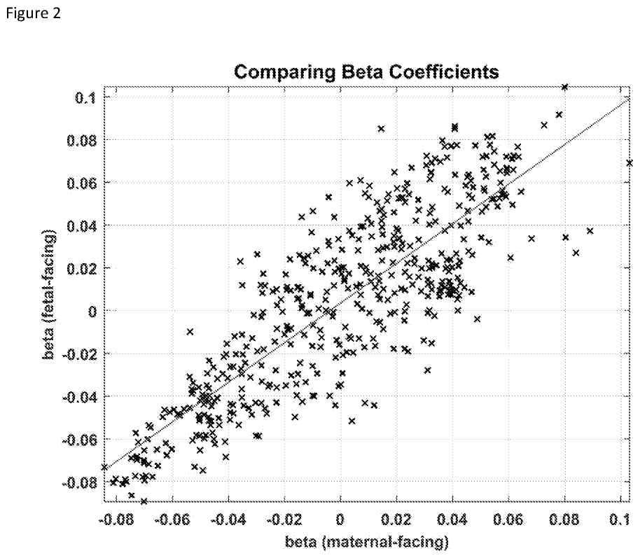

In the pilot study, 57 maternal-facing placenta samples and 57 fetal-facing placenta samples were analysed in a single batch. Pooled placenta quality controls (QCs) were inserted in the run once every 10 study samples. The percentage coefficient of variation was computed on the basis of the QCs for all lipid species. Lipid species with a % CV of more than 50% were omitted from subsequent analysis. A polynomial-fit (order 7) correction based on quality controls was employed to minimize the effects of drifts in sensitivity in the mass spectrometer over the batch, this approach is similar to the QC-RLSC protocol that is described in Dunn et al. 2011 Nat Protocols 2011; 6(7): 1060-83. Multiple linear regression was used to determine the association of placenta lipid species with antenatal EPDS scores. Linear regression was run separately for the maternal-facing placenta samples and the fetal-facing placenta samples and the beta coefficients from both analyses were compared using Pearson's correlation, and is shown as FIG. 2.

In the main placenta experiment, 1048 maternal-facing placenta samples were analysed in targeted lipidomic analysis run across 7 batches. Pooled placenta quality controls (QCs) were also inserted once every 10 study samples in the run. The percentage coefficient of variation was computed on the basis of the QCs for all lipid speciesLipid species with a % CV of more than 50% were omitted from subsequent analysis. Within each batch, a polynomial-fit (order 7) correction based on quality controls was employed to minimise the effects of drifts in sensitivity in the mass spectrometer over the course of the run. After the application of the polynomial-fit correction, the measurements of the lipid species in all samples of each batch were aligned by multiplying the lipid levels by the reciprocal of the ratio of the median lipid measurement in the QCs of each batch to the median level of the lipid in the QCs across all batches, this approach is also commonly known as median centeringLipid species with a correlation coefficient of more than 0.4 or less than −0.4 with respect to the run order were deemed to have residual batch effects and were omitted from subsequent analysis. 470 lipid species passed quality filtering and were analysed in downstream statistical analysis.

Statistical analyses were conducted using scripts and libraries written in MATLAB® R2017a. Multiple adjusted linear regression was used to determine:
(i) the association of placental lipids with antenatal EPDS,
(ii) the association of plasma phosphatidylcholine fatty acids with antenatal EPDS
(iii) association of antenatal EPDS with CBCL scales in the offspring at 48 months and
(iv) the association of antenatal depressive mood related placental lipids with CBCL scales in the offspring at 48 months.

Placental lipid species and plasma fatty acids were log 10-transforrned and z-score standardized before incorporation into the model. CBCL scores were utilized in their t-score format.

Covariates such as pre-pregnancy BMI were also log 10-transforrned and z-score standardized before use.

Results

High Correlation of Lipid Profiles Between Maternal and Fetal Sides of Placenta

In a pilot study (N=57) comparing the standardised beta-coefficients of linear regression analysis based on the maternal and fetal-facing placenta, as shown in FIG. 2, we found that the measurements of lipids in both sets of samples were highly correlated (rho=0.843, p=3e−129) and that measurements of the maternal-facing placenta samples would be sufficiently representative of the shared placental environment between the mother and offspring.

Antenatal Depression Associates with Distinct Lipid Profile of Placenta

In the main lipidomics study we analysed placenta from 186 women with low (n=70, EPDS score<2), or high (n=116, EPDS score>10) levels of antenatal depressive symptoms at 26 weeks gestation. The multiple linear regression analysis adjusted for fish-oil consumption and pre-pregnancy BMI identified 17 lipid species in placenta (Table 2, below) to be significantly (FDR corrected p<0.05) associated with EPDS scores. These lipid species included: phosphatidylcholine (PC), phosphatidylcholine plasmalogen (PC(P)), phosphatidylethanolamine (PE), phosphatidylethanolamine plasmalogen (PE(P)), alkylphosphatidylethanolamine (PE(O)), phosphatidylinositol (PI), phosphotydylserine (PS) and sphingomyelin (SM) with the majority of lipid species (94%) being phospholipids.

There was a distinct preponderance for phospholipids containing LC-PUFAs i.e., EPA (C20:5, n-3), DPA (C22:5, n-3), DHA (C22:6, n-3) or AA (C20:4, n-6). 76% of the significant lipid species contained at least one form of the stated LC-PUFAs, suggesting specificity in the associations of phospholipids containing LC-PUFAs with antenatal depressive symptoms. This is shown in Table 2 below.

TABLE 2
List of placental lipids significantly associated (FDR p < 0.05)
with EPDS at 26 weeks as determined by multiple adjusted linear regression.

| Placental | Fatty Add | Beta Coefficient | Lower | Upper | FDR- |
|---|---|---|---|---|---|
| Lipids | Composition | (standardised) | Bound | Bound | corrected p |
| PE(P-17:0/20:4)(a) | PE(P-17:0/20:4, n-6) | −0.587 | −0.873 | −0.302 | 0.024 |
| PE(P-17:0/22:6)(a) | PE(P-17:0/22:6, n-3) | −0.569 | −0.850 | −0.289 | 0.024 |
| PE(O-36:5) | PE(O16:1/20:4, n-6), | −0.540 | −0.825 | −0.256 | 0.041 |
| | PE(O-16:0/20:5, n-3) | | | | |
| PC 35:5 | PC(15:0/20:5, n-3) | −0.499 | −0.775 | −0.222 | 0.046 |
| PE(P-16:0/20:5) | PE(P-16:0/20:5, n-3) | −0.503 | −0.784 | −0.222 | 0.046 |
| PC 37:6 | PC(15:0/22:6, n-3) | −0.495 | −0.775 | −0.215 | 0.046 |
| PC 34:5 | PC(P-14:0/20:5, n-3) | −0.483 | −0.762 | −0.204 | 0.046 |
| PS 40:5 | PS(18:0/22:5, n-3) | −0.497 | −0.787 | −0.207 | 0.046 |
| PE(P-18:0/20:5) | PE(P-18:0/20:5, n-3) | −0.480 | −0.761 | −0.200 | 0.046 |
| PE 40:5(a) | PE(18:0/22:5, n-3) | −0.487 | −0.776 | −0.198 | 0.046 |
| PI 35:2 | PI(17:0/18:2) | −0.484 | −0.771 | −0.197 | 0.046 |
| SM 35:1(b) | SM(17:1/18:0) | −0.471 | −0.753 | −0.188 | 0.046 |
| PC 33:2 | PC(15:0/18:2) | −0.464 | −0.744 | −0.184 | 0.046 |
| PE(P-16:0/22:5)(a) | PE(P-16:0/22:5, n-3) | −0.479 | −0.769 | −0.189 | 0.046 |
| PC(P-36:5) | PC(P-16:0/20:5, n-3) | −0.464 | −0.745 | −0.182 | 0.046 |
| PC 39:5(a) | PC(17:0/22:5, n-3) | −0.465 | −0.750 | −0.180 | 0.046 |
| PI 40:5(a) | PI(18:0/22:5, n-3) | −0.464 | −0.749 | −0.179 | 0.046 |

Omega fatty acids levels are longitudinally associated with antenatal depressive symptoms To determine if the placental lipid signatures were also observed at an earlier timepoint in pregnancy (26 wks) we assessed a selection of fatty acids in maternal plasma and identified three fatty acids in antenatal plasma phosphatidylcholine to be associated with antenatal depressive symptoms at 26 weeks. DPA (beta=−0.469, p=0.001) and EPA (beta=−0.389, p=0.005) were inversely associated with antenatal depressive symptoms while dihomo-gamma-linolenic acid (C2013, n-6) or DGLA (beta=0.331, p=0.026) was positively associated with antenatal depressive symptoms. DHA (beta=−0.213, p=0.154) and AA (beta=0.121, p=0.43) in antenatal plasma were not significantly associated with antenatal depressive symptoms, although the direction of association of DHA with antenatal depressive symptoms was consistent with that of DPA and EPA (Table 3), below.

TABLE 3
Cross-sectional association of fatty acids in
plasma phosphatidylcholine with EPDS at 26 weeks
as determined by multiple linear regression.

| Fatty Acids in Plasma | Beta Coefficient | Lower | Upper | |
|---|---|---|---|---|
| Phosphatidylcholine | (standardised) | Bound | Bound | p |
| C22:5(n-3) | −0.469 | −0.744 | −0.195 | 0.001 |
| C20:5(n-3) | −0.389 | −0.660 | −0.118 | 0.005 |
| C20:3(n-6) | 0.331 | 0.041 | 0.620 | 0.026 |
| C16:1(n-7) | 0.295 | −0.001 | 0.592 | 0.052 |
| C22:4(n-6) | −0.266 | −0.561 | 0.030 | 0.079 |
| C18:1(n-9) | 0.265 | −0.031 | 0.562 | 0.081 |
| C22:6(n-3) | −0.213 | −0.504 | 0.079 | 0.134 |
| C20:0 | −0.209 | −0.505 | 0.087 | 0.168 |
| C18:3(n-6) | −0.156 | −0.455 | 0.143 | 0.308 |
| C20:1(n-9) | −0.135 | −0.434 | 0.163 | 0.376 |
| C20:4(n-6) | 0.121 | −0.178 | 0.420 | 0.430 |
| C18:3(n-3) | −0.106 | −0.404 | 0.191 | 0.484 |
| C18:0 | −0.101 | −0.398 | 0.195 | 0.504 |
| C22:0 | −0.091 | −0.390 | 0.208 | 0.551 |
| C20:2(n-6) | 0.061 | −0.232 | 0.354 | 0.683 |
| C18:2(n-6) | −0.050 | −0.350 | 0.249 | 0.743 |
| C20:4(n-3) | −0.036 | −0.328 | 0.255 | 0.808 |
| C16:0 | 0.030 | −0.325 | 0.266 | 0.845 |
| C18:1(n-7) | −0.016 | −0.313 | 0.282 | 0.918 |
| C14:0 | 0.003 | −0.295 | 0.300 | 0.985 |

Antenatal Depressive Symptoms and Child Socio-Emotional Problems

In multiple linear regression analysis adjusted for maternal education, gestational age and sex, we identified antenatal EPDS scores to be significantly associated (p<0.05) with 13 out of the 15 CBCL scales (Table 4).

TABLE 4
Association of antenatal EPDS at 26 weeks with
CBCL scales at 48 months in the offspring as determined
by multiple adjusted linear regression.

| CBCL scales | Beta | Lower | Upper | |
|---|---|---|---|---|
| (t-scores) | Coefficient | Bound | Bound | p |
| DSM Anxiety Problems | 6.44 | 3.61 | 9.28 | 0.00002 |
| Internalizing Problems^{†} | 9.09 | 4.95 | 13.23 | 0.00004 |
| Total Problems | 8.68 | 4.56 | 12.80 | 0.0001 |
| Anxious/Depressed* | 4.67 | 2.14 | 7.20 | 0.0004 |
| Emotionally Reactive* | 4.28 | 1.81 | 6.75 | 0.001 |
| Externalizing Problems^{‡} | 6.11 | 2.19 | 10.02 | 0.003 |
| DSM Affective Problems | 4.18 | 1.43 | 6.94 | 0.004 |
| Sleep Problems^{‡} | 3.50 | 1.14 | 5.86 | 0.004 |
| DSM Pervasive | 4.01 | 0.95 | 7.08 | 0.012 |
| Developmental Problems | | | | |
| Somatic Complaints* | 3.37 | 0.67 | 6.06 | 0.016 |
| Attention Problems^{†} | 2.81 | 0.51 | 5.11 | 0.018 |
| Aggressive Behaviour^{†} | 2.44 | 0.26 | 4.61 | 0.030 |
| Withdrawn* | 3.62 | 0.36 | 6.87 | 0.031 |
| DSM Oppositional Defiant | 1.25 | −0.86 | 3.36 | 0.249 |
| Problems | | | | |
| DSM ADHD | 0.92 | −1.09 | 2.92 | 0.373 |

*components of internalising problems score
^{†}components of externalising problems score

The associations were all positive implying that higher EPDS scores are associated with increased socio-emotional problems in the offspring. Only DSM Oppositional Defiant Problems and DSM ADHD scales were not significantly associated with antenatal EPDS scores.

Placental Lipids Linked with Antenatal Maternal Depression are Also Associated with Child Behavioral Outcomes

We next assessed the association of 17 antenatal depression linked lipid species with CBCL scores at 48 months and identified 8 lipids to show an association (FDR corrected p<0.05) with five CBCL scales as shown in Table 5, below.

TABLE 5
Association of placental lipid levels with CBCL scales at 48 months in
the offspring as determined by multiple adjusted linear regression.

| | Attention | | DSM Pervasive | | Emotionally | | DSM Anxiety | | DSM | |
|---|---|---|---|---|---|---|---|---|---|---|
| Placental | Problems | | Developmental Problems | | Reactive | | Problems | | ADHD | |
| Lipids | Beta | p | Beta | p | Beta | p | Beta | p | Beta | p |
| PC 33:2 | −1.128 | 0.058 | −1.006 | 0.207 | −0.738 | 0.251 | −1.070 | 0.146 | −0.908 | 0.081 |
| PC 34:5 * | −0.004 | 0.995 | −1.640 | 0.039 | −0.854 | 0.185 | −1.223 | 0.097 | −0.155 | 0.768 |
| PC 35:5 * | −0.657 | 0.290 | −1.781 | 0.030 | −0.918 | 0.168 | −1.396 | 0.067 | −0.661 | 0.222 |
| PC 37:6 | −1.079 | 0.069 | −1.414 | 0.073 | −0.956 | 0.135 | −1.414 | 0.053 | −0.917 | 0.076 |
| PC 39:5(a) [symbol] | −1.137 | 0.054 | −1.096 | 0.164 | −1.046 | 0.099 | −1.086 | 0.136 | −1.039 | 0.043 |
| PC(P-36:5) | −0.446 | 0.445 | −0.983 | 0.206 | −0.368 | 0.558 | −0.553 | 0.443 | −0.446 | 0.381 |
| PE 40:5(a) ^{†} | −1.672 | 0.004 | −1.153 | 0.147 | −1.405 | 0.027 | −1.330 | 0.070 | −0.922 | 0.075 |
| PE(O-36:5) | 0.005 | 0.994 | 0.875 | 0.265 | 0.573 | 0.366 | 0.951 | 0.190 | 0.095 | 0.854 |
| PE(P-16:0/20:5) * | −1.268 | 0.031 | −0.901 | 0.253 | −1.099 | 0.083 | −0.520 | 0.477 | −0.978 | 0.057 |
| PE(P-16:0/22:5)(a) | −0.918 | 0.116 | −0.070 | 0.929 | −0.699 | 0.267 | −0.619 | 0.392 | −0.403 | 0.431 |
| PE(P-17:0/20:4)(a) [symbol] | −1.422 | 0.020 | −1.434 | 0.081 | −0.341 | 0.609 | 0.053 | 0.945 | −0.769 | 0.154 |
| PE(P-17:0/22:6)(a) ^{‡} | −1.250 | 0.038 | −1.101 | 0.173 | −0.664 | 0.310 | −0.539 | 0.472 | −0.876 | 0.097 |
| PE(P-18:0/20:5) | −0.436 | 0.466 | −0.896 | 0.260 | −0.604 | 0.347 | −0.589 | 0.424 | −0.448 | 0.390 |
| PI 35:2 | −0.649 | 0.275 | −0.295 | 0.710 | −0.072 | 0.910 | −0.398 | 0.588 | −0.623 | 0.229 |
| PI 40:5(a) ^{†} | −1.145 | 0.052 | −1.550 | 0.048 | −1.206 | 0.057 | −1.611 | 0.026 | −0.905 | 0.078 |
| PS 40:5 | −0.972 | 0.103 | −1.014 | 0.202 | −0.564 | 0.381 | −0.846 | 0.251 | −0.470 | 0.367 |
| SM 35:1(b) | −0.450 | 0.449 | −0.975 | 0.217 | −0.032 | 0.961 | −0.741 | 0.311 | −0.996 | 0.053 |

* contains EPA (C20:5, n-3)
† contains DPA (C22:5, n-3)
‡ contains DHA (C22:6, n-3) [symbol]
contains AA (C20:4, n-6) [symbol]
indicates data missing or illegible when filed

These associations persisted after adjusting for maternal EPDS status suggesting a direct association between the lipid and the socio-emotional outcome. PE 40:5(a) which contains DPA, PE(P-16:0/20:5) which contains EPA, PE(P-17:0/20:4)(a) which contains AA, and PE(P-17:0/22:6)(a) which contains DHA were significantly associated with the Attention Problems scale.

PC 34:5 which contains EPA, PC 35:5 which contains EPA and PI 40:5 which contains DPA were significantly associated with the DSM Pervasive Developmental Problems scale.

PE 40:5(a) which contains DPA was significantly associated with the Emotionally Reactive scale.

PI 40:5(a) which contains DPA was significantly associated with DSM Anxiety Problems scale.

PC 39:5(a) which contains DPA was significantly associated with DSM ADHD scale.

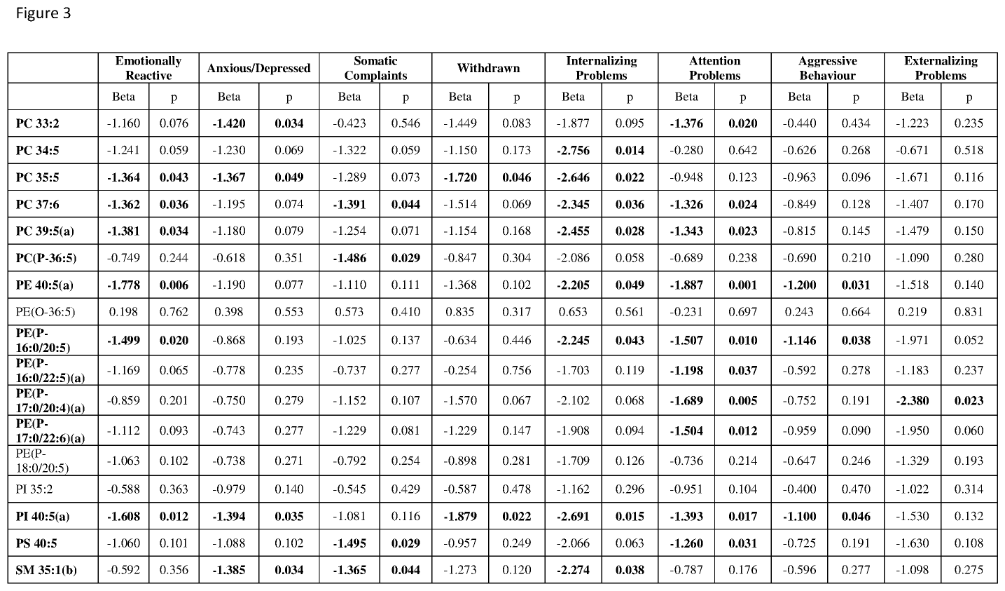

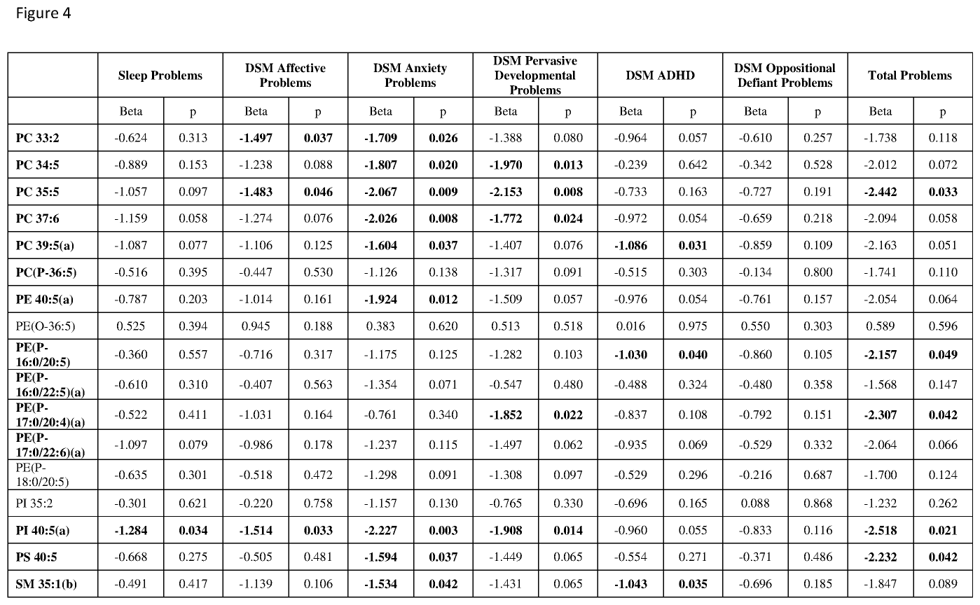

EPA and DPA contributed to the fatty acid composition of three significant lipid species each while DHA and AA contributed to fatty acid composition of one significant lipid species each (summarized in Table 5). FIGS. 3 and 4 summarize the linear regression results for all 15 CBCL scales without including EPDS as a covariate.

### Discussion

We analysed the placental lipidome of 186 subjects with varying levels of depressive symptoms assessed at 26 weeks of pregnancy. A total of 470 lipid species in placenta were considered in statistical analysis. Phospholipids containing polyunsaturated fatty acids such as DHA, DPA, EPA and AA had the strongest associations with antenatal depressive symptoms. A cross-sectional association analysis of second trimester (26 weeks) maternal plasma phosphatidylcholine fatty acids and EPDS replicated the placental findings for EPA and DPA. For DHA, the directionality of association with EPDS was the same between placenta and plasma, though the association passed significance only in placenta.

In adults, DHA and DPA can be synthesised from linoleinic acid (18:3), but are primarily derived from the diet. The placenta and fetus lack the desaturase enzymes required to synthesize the LC-PUFAs and are therefore entirely reliant on the maternal supply, thus LC-PUFAs are considered essential fatty acids for the fetus. During pregnancy, the essential fatty acids in the maternal circulation available for transplacental transfer to the fetus are derived from the maternal diet, from maternal metabolism of precursor fatty acids and the breakdown of adipose tissue stores. The importance of the association of these LC-PUFAs with antenatal maternal depressive symptoms is apparent in the finding that the level of circulating phosphatidylcholine containing LC-PUFAs in the mother is strongly associated with those in the placenta. This is consistent with the general notion that the concentration of maternal circulating free fatty acids is a main determining factor of the umbilical cord free fatty acid concentration in uncomplicated pregnancies. Although not all phospholipids were profiled in antenatal maternal plasma, phosphatidylcholine, the most abundant phospholipid class provides a reasonable gauge of the LC-PUFAs levels in plasmaphospholipids.

Fetal free fatty acid levels are dependent upon placental transfer primarily determined by maternal diet, and are important for neurodevelopment. Observational studies suggest that consumption of fish rich in omega-3 LC-PUFAs during pregnancy is positively associated with neurodevelopmental outcomes, including child behavioural outcomes.

We used a prospective longitudinal study design to determine if maternal circulating lipids, antenatal EPDS scores together with the placental lipid profile predicts neurodevelopmental outcomes in the offspring, focusing on measures of socio-emotional problems that are known to associate with antenatal maternal depression and predict the later risk for psychopathology in the child. We identified that lower levels of phospholipid species containing EPA, DPA, DHA and AA fatty acids in the placenta were linked to reduced levels of EPA, DPA in plasma phosphatidylcholine during pregnancy and that these phospholipids were significantly associated with poorer socio-emotional outcomes on multiple CBCL scales at 48 months of age. The LC-PUFAs that drive the association with CBCL scales are omega-3 EPA (2025, n-3), DPA (22:5, n-3), DHA (2226, n-3) and omega-6 arachidonic acid (20:4, n-6). These findings are consistent with those using measures of dietary reports documenting a positive association between LC-PUFA intakes and better neurodevelopmental outcomes 23. Our findings suggest that antenatal maternal nutritional status serves as an in utero factor that drives the association between maternal depressive symptoms and neurodevelopment outcomes in the offspring, including phenotypes that predict the later risk for depression.

This conclusion is consistent with the premise that fetal nutrition shapes fetal neurodevelopment and later mental health outcomes. There is evidence from developmental neuroscience for the biological plausibility of an association between fetal lipids and neurodevelopment. Fatty acids are required for optimal growth, development, and function of brain tissue. The third trimester of pregnancy features dynamic structural neural growth of the fetal brain, which coincides with a massive increase in the selective accumulation of DHA and other LC-PUFAs, which are preferentially taken up and transferred across the placenta. Studies with model systems show that perinatal brain DHA accrual is required for normal neurotrophic factor expression, neurite outgrowth, neurogenesis and migration and neuronal differentiation and the function neurotransmitter systems as well as transmitter receptor function through influences on membrane fluidity.

These findings provide the first report suggesting that an in utero nutritional status deficient in placental phospholipids containing LC-PUFAs, relates to both antenatal maternal depressive symptoms and later childhood socio-emotional problems. Our study benefits from a prospective, longitudinal design with direct measures of maternal nutrient status and placental lipidomic profiling. We postulate that a low maternal omega-3 level may limit the ability of the expecting woman to cope with prevailing psychological stressors leading to antenatal depressive symptoms during pregnancy, whilst at the same time being unable to meet the demands of the fetus who is competing for the limited LC-PUFA resource, resulting in suboptimal fetal brain development and subsequently poor child metal health outcomes.

Our study design relies on the EPDS, rather than direct clinical assessment of depression; we therefore refer to the findings as reflecting an association between the number of depressive symptoms and lipidomic profiles. However, the EPDS is a highly validated screening tool for perinatal depression. Importantly, our sample included women with EPDS scores largely falling in the high range of sub-clinical to lower range of clinical scores, and were thus not unique to more severe cases of maternal depression.

Likewise, our previous neuroimaging studies suggest that the number of depressive symptoms associates with fetal cortico-limbic development and that this association is observed in non-clinical community samples, suggesting that maternal mental health influences fetal neurodevelopment across the population. Similarly, while CBCL scores originate from parental questionnaires rather than formal neurodevelopmental assessments they are validated and showed high reliability in Singaporean children. The objective measure of placental lipids is a major strength of our study, however further analysis of cord blood can provide a more accurate quantification of true transplacental transfer of lipids.

This study provides new insights into the biological pathways by which maternal emotional wellbeing influences fetal neurodevelopment and the later risk for psychopathology. Our results suggest a potential utility of nutritional approaches in the prevention of risk for later offspring psychopathology among pregnant women with depressive symptoms. This is particularly pertinent in light of the reluctance of pregnant women to adopt pharmacological interventions, including anti-depressant medications. We note, however, that while diet is inevitably important in determining fatty acid availability for the fetus, there might also influences of genetic variations that affect free fatty acid synthesis, metabolism and transport across the placenta that need to be considered in tandem with nutritional supplementation efforts.

The limitations of previous studies have been:
Nutritional intervention not attempted before omega 3 fatty acid demand peaks (i.e. early in pregnancy, or at preconception). (J Matern Fetal Neonatal Med. 2011 May; 24(5): 680-686.)
Interventions used DHA or a combination of DHA+ EPA, which is less potent than DPA. Docosapentaenoic acid (DPA), an elongated version of EPA, has not been studied as extensively as EPA or DHA due to limited availability of its pure form. In fact, DPA is only present in many fish oil supplements in quantities of 3% or less. However, DPA is gradually being recognized for its role in human health. While EPA retro-converts to DPA, the conversion can be influenced by omega-6 fatty acids in the diet, alcohol consumption and smoking, other enzyme co-factors and genetic factors thus direct supplementation with DPA will enable maximum benefits to be reaped.
The longitudinal cohorts primarily used food frequency questionnaire to evaluate fish and omega-3 consumption in the perinatal period and not the more reliable blood or placental lipid measures ((J Matern Fetal Neonatal Med. 2011 May; 24(5): 680-686).

1. A method of enhancing the low mood of a peri-partum woman comprising administering an effective amount of docosapentaenoic acid (DPA) to the woman.

2. A method of enhancing the socio-emotional outcome of a child comprising antenatally administering to the child's mother an effective amount of at least one polyunsaturated fatty acid (PUFA) selected from the group consisting of DPA, eicosapentaenoic acid (EPA), arachidonic acid (AA) and docosahexaenoic acid (DHA).

3. A method according to claim 2 wherein the enhancing is related to attention problems.

4. A method according to claim 2 wherein the enhancing is related to a pervasive developmental problem and the at least one PUFA is EPA or DPA.

5. A method according to claim 2 wherein the enhancing is related to emotional reactivity problems and the PUFA is DPA.

6. A method according to claim 2 wherein the enhancing is related to anxiety.

7. A method according to claim 2 wherein the enhancing is related to at least one component selected from the group consisting of: inattention, hyperactivity, and impulsivity, and the PUFA is DPA.

8. A method according to claim 1 wherein the PUFA is DPA and the daily dose is from 1 to 50 mg, preferably from about 2 to 25 mg, and more preferably from 5 to 10 mg.

9. A method according to claim 8 wherein the daily dose is 5 mg.

10. A method according to claim 8 wherein the daily dose is 10 mg.

11. A maternal nutritional supplement comprising DPA in a tablet or capsule form.
